(19) United States
(12) Patent Application Publication
(10) Pub. No.: US 2013/0244334 A1
(43) Pub. Date: Sep. 19, 2013

(54) DEVICE FOR THE DETECTION AND/OR ELECTRICAL QUANTIFICATION OF ORGANOPHOSPHORUS COMPOUNDS BY MEANS OF MOLECULAR IMPRINTING

(71) Applicants: Simon Clavaguera, Alenya (FR); Alexandre Carella, Mazeres Lezons (FR); Caroline Celle, Firminy (FR); Jean-Pierre Simonato, Sassenage (FR)

(72) Inventors: Simon Clavaguera, Alenya (FR); Alexandre Carella, Mazeres Lezons (FR); Caroline Celle, Firminy (FR); Jean-Pierre Simonato, Sassenage (FR)

(73) Assignee: COMMISSARIAT A L'ENERGIE ATOMIQUE ET AUX ENERGIES ALTERNATIVES, Paris (FR)

(21) Appl. No.: 13/809,092

(22) PCT Filed: Jul. 4, 2011
(86) PCT No.: PCT/FR2011/051564

§ 371 Date: May 22, 2013

(30) Foreign Application Priority Data
Jul. 8, 2010 (FR) 1002871

(51) Int. Cl. G01N 33/00

(52) U.S. Cl. CPC G01N 33/00
USPC 436/104; 422/82.02; 526/259

(57) ABSTRACT

The present invention relates to a method and device for the detecting and/or electrically quantifying organophosphorus compounds as a gas or solution. The device includes: an electrical device including a source electrode and a drain electrode, separated by a semiconductor material; and a device for detecting positive charges between the two electrodes. One surface of the semiconductor material is in contact with a polymer material having at least one receptor group including: 1) a function X capable of reacting with the detectable organophosphorus compound, to form an intermediate grouping —Z; and 2) a function Y capable of reacting with —Z to form a positively charged ring. The polymer material includes molecular imprint cavities, with geometrical and chemical configuration allowing the detectable organophosphorus compounds to penetrate into said polymer material. The invention can be used in the field of detecting and/or quantifying organophosphorus compounds.

The present invention relates to a method and a device for the electrical detection and/or quantification of organophosphorus compounds present in gaseous form or in solution.

Organophosphorus compounds are molecules constituted of a phosphate atom to which various chemical groups are bonded, the nature of said groups determining the exact properties of the compound. Organophosphorus compounds, which were the subject of intensive research on combat gases during and after the Second World War, which resulted in the development of the gases sarin, soman, tabun, cyclosarin, GV, VX, VE, VG and VM, and of molecules which simulate the action of neurotoxic organophosphorus compounds, of the type DFP (diisopropyl fluorophosphate), DCP (diethyl chlorophosphate) and DMMP (dimethyl methylphosphonate), are today mainly used in agriculture as insecticides or herbicides. Among the large variety of organophosphorus compound-based pesticides that exist, mention may in particular be made of parathion, malathion, methyl parathion, chlorpyrifos, diazinon, dichlorvos, phosmet, tetrachlorvinphos and methyl azinphos.

The mode of action of organophosphorus compounds is based on the affinity of the latter for an enzyme involved in nerve impulse transmission: cholinesterase. Organophosphorus compounds in fact have the property of binding particularly strongly and stably to the active site of this enzyme. Once bound, the organophosphorus compound prevents the cholinesterase from degrading acetylcholine, a neurotransmitter released at the neuronal synapses during neuronal excitation: through a lack of degradation of acetylcholine to inactive choline and acetate, the cholinergic receptors are constantly stimulated, which may lead to paralysis and result in death.

Since the mechanism involved in nerve impulse transmission which is blocked by organophosphorus compounds is identical throughout the animal kingdom, the organophosphorus compounds used as insecticides are not only toxic to insects, but also to any animal, including humans. For this reason, despite their relatively good biodegradability which has enabled them to replace organochloro compound-based insecticides which have a poor biodegradability, their often much greater toxicity requires particular precautions for use. Furthermore, since these insecticides are among those most widely used, not only in agriculture by individuals working in the industry, but also by private individuals, the detection and the quantitative determination of organophosphorus compounds represent a public health interest and would also be particularly useful for the agri-food industry.

In addition, despite being banned in 1997, combat gases are still a threat. On the one hand, the production of combat gases of the sarin, soman or VX type is easy and, on the other hand, since these compounds are odorless and colorless, they can be inhaled without the individual realizing it. The immediate detection of the presence of such gases therefore represents a major issue for protecting soldiers in the combat zone, but also civilian populations in the face of the risk of terrorism. These threats are clearly established, especially since the deadly sarin attack carried out by the terrorist group Aum Shinrikyo in the Tokyo subway in 1995.

Many methods and devices for detecting organophosphorus compounds have been developed.

Thus, chemical sensors sensitive to organophosphorus gases, most of which operate on the “electronic nose” principle, are sold.

U.S. Pat. No. 5,571,401, for example, describes a chemical sensor constituted of arrays comprising a resistor composed of nonconductive polymers and of conductive materials: when a chemical molecule comes into contact with the conductive materials, a difference in resistance is then detected. These sensors have the drawback of not being very specific and of being subject to false positives. Sensors based on two-dimensional grids of carbon nanotubes have also been developed (WO 2006/099518), but, although they are very sensitive and detect a large number of molecules, they do not make it possible to obtain a specific and selective response for organophosphorus compounds.

Recently, selective detection methods for organophosphorus compounds in solution, based on measuring fluorescence, have been described. Zhang et al. have shown that, in the presence of a molecule having an amine in spatial proximity to a primary alcohol and a non-planar, flexible and weakly conjugated chromophore, organophosphorus compounds react with the primary alcohol, the phosphate obtained enabling cyclization by intramolecular nucleophilic substitution, stabilizing the chromophore in the plane and resulting in an increase in fluorescence (S.-W. Zhang and T. M. Swager, J. Am. Chem. Soc., 2003, 125, 3420-3421). This cyclization therefore enables detection, by fluorescence, of the organophosphorus agents in solution. The team of J. Rebek, Jr. subsequently showed that some Kemp's acid derivatives comprising a primary alcohol close to a tertiary amine are also good candidates for detecting neurotoxic organophosphorus species (T. J. Dale & J. Rebek, Jr., J. Am. Chem. Soc., 2006, 4500-4501). As S.-W. Zhang et al. had previously shown, when the primary alcohol reacts with an organophosphorus compound, the phosphate obtained enables cyclization by intramolecular nucleophilic substitution and the obtaining of a quaternary ammonium according to the reaction shown in the scheme below:

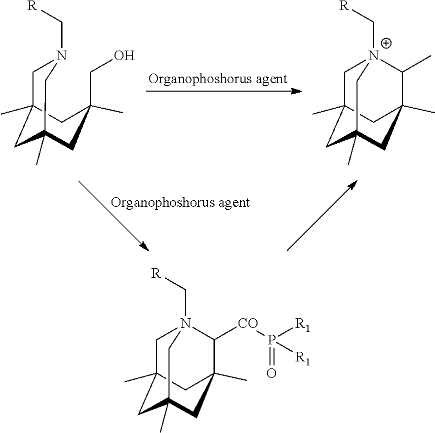

Rebek et al. then developed Kemp's acid derivatives in which R is a fluorophore. In its open form, the fluorescence is prevented by a photoinduced electron transfer due to the amine. The cyclization abolishes this electron transfer and increases the fluorescence of the molecule. A 5-second exposure of a filter paper impregnated with Kemp's acid coupled to a fluorophore in an atmosphere comprising 10 ppm of DFP enables the detection of this organophosphorus agent by reading the fluorescence under a UV lamp. Although this detection is specific, it has various drawbacks. First of all, it must be carried out in a low light environment, which requires at least a cover. Furthermore, it requires the use of a UV lamp and of a photon counter, which increases the electricity consumption and the bulk and therefore reduces the portable nature of the device for detecting and/or quantifying organophosphorus compounds. It cannot therefore always be used on premises where the presence of organophosphorus compounds is to be detected in real time.

Methods based on the use of a biosensor comprising enzymes which have an affinity for organophosphorus compounds, for example cholinesterase, have also been developed. In PCT Application WO 2004/040004, unicellular algae which express acetylcholinesterase at the membrane have been used to test for the presence of organophosphorus compounds in an aqueous medium, the percentage inhibition of acetylcholinesterase being correlated with the concentration of organophosphorus compounds in the aqueous liquid tested. This method requires the solubilization of the organophosphorus compounds prior to the detection thereof.

Recently, a method based on the use of molecularly imprinted polymers was developed for detecting explosives, by fluorescence.

Thus, patent application EP 2 866 429 describes chemical sensors intended for detecting explosives, especially nitroaromatic compounds, comprising a fluorescent material capable of forming a charge-transfer complex with the type of molecule to be detected and means for measuring the variation of fluorescence of said material. These sensors also comprise a filter containing a polymer material comprising cavities referred to as “molecularly imprinted cavities”, the geometric and chemical configuration of which is defined so as to fix the type of molecule to be detected. Generally, the polymers referred to as molecularly imprinted polymers (MIPs) are robust biomimetic systems that make it possible to selectively capture a given molecule type. Just like biological receptors, MIPs benefit from a great affinity and a good selectivity for a family of particular molecules. A priori, MIPs can be devised in the image of any molecule or family of molecules as a function of their size or of the chemical functions that they bear. Thus, the synthesis of MIPs can be envisaged for any chemical family. Due to their highly crosslinked chemical structure, MIPs have a very good thermal and chemical stability.

However, the selectivity of these chemical sensors remains limited to steric aspects, namely the size of the cavities of the molecularly imprinted polymer. In addition, these chemical sensors, the transduction of which is of fluorescent nature, require a light source and a light detector and also a power supply and complex associated electronics. These sensors are therefore expensive and difficult to miniaturize.

One method for the selective detection of organophosphorus compounds has furthermore been described in patent application EP 2154525. This describes a device for detecting and/or quantifying organophosphorus compounds comprising an electrical device comprising a source electrode and a drain electrode separated by a semiconductor material and a device for detecting the variation of the positive charges between the two electrodes. The electrodes or the semiconductor material are grafted by receptor molecules, capable of reacting with the organophosphorus compounds to be detected, in order to form a positively charged ring.

However, such devices pose problems of stability over time. Indeed, the semiconductor materials, and more particularly carbon nanotubes, are often sensitive to oxygen, moisture and also to atmospheric interferents. In addition, the latter may pose problems of background noise liable to be detrimental to the sensitivity of the detection device.

In summary, the devices for detecting organophosphorus compounds that are currently available have the drawback either that they are not selective for organophosphorus compounds, or that they can be used only for testing a sample in solution, or that they require a fluorescence reader and a low light intensity environment or else they have stability problems. The development of a rapid, effective and specific method for the selective detection of organophosphorus compounds both in gaseous form and in solution, in any medium, irrespective of the light intensity thereof, and also the development of a compact and readily portable device for simple, rapid and selective detection therefore still represent major issues in the protection of soldiers in the combat zone and of civilian populations exposed to the risk of terrorism and more broadly for the detection of organophosphorus pesticides.

It has now been demonstrated that by combining a semiconductor material with a sensitive material based on a polymer material of the molecularly imprinted polymer (MIP) type grafted by receptor molecules, the molecularly imprinted polymer and the receptor molecules both being selective for the organophosphorus compounds to be detected, it was possible to simultaneously considerably increase the selectivity, the sensitivity and the service life of the chemical sensor.

The result observed by the inventors is all the more surprising since the presence of the molecularly imprinted material would have been expected to have the effect of reducing the accessibility of the receptor molecules and therefore the sensitivity and/or the chemical selectivity of the sensor. In addition, this molecularly imprinted material would have also been expected to have a “screening effect”, i.e. to reduce the detection of positive charges at the semiconductor material, and therefore the sensitivity of the sensor.

The molecules referred to as “receptor molecules” according to the invention are molecules that are sensitive and specific for the organophosphorus analytes to be detected, which are capable of generating an electric charge after reaction with the organophosphorus compounds. This charge formation then modifies the electrical properties of the semiconductor material. The detection is achieved by recording, over time, the change in the electrical properties of the semiconductor material.

More specifically, it has been shown that this polymer material grafted by receptor groups made it possible to increase the number of grafted receptor groups and therefore the sensitivity of the material.

In addition, it has been demonstrated that the molecularly imprinted polymer, by allowing only certain species to penetrate into the sensitive material and thus to react with the grafted receptor groups, advantageously made it possible to increase the selectivity of the sensitive material while protecting the semiconductor material from many interferents. This polymer material therefore acts as a barrier layer, for protecting the semiconductor material, that makes it possible to limit the false positives commonly encountered when working with a bare or grafted semiconductor material, but also as a sorting device by preferentially letting through the molecules of interest. This polymer material thus makes it possible to obtain a two-fold selectivity, both steric and chemical. Specifically, it makes it possible to detect molecules which have, on the one hand, a structure analogous to the imprint used to form the MIP (shape, size, chemical functions) and, on the other hand, which are capable of reacting with the receptor incorporated in the MIP polymer.

Thus, a first subject of the invention relates to a device for detecting and/or quantifying at least one organophosphorus compound comprising:
an electrical device comprising a source electrode and a drain electrode separated by a semiconductor material, and
a device for detecting positive charges between the two electrodes.

Said device being characterized in that one of the surfaces of the semiconductor material is in contact with an intermediate polymer material bearing at least one receptor group comprising:
a function X capable of reacting with the organophosphorus compound to be detected in order to form an intermediate group —Z, X being chosen from —CH₂OH, —CH₂NH₂, —CH₂SH; and
a function Y capable of reacting with said group —Z in order to form a ring bearing a positive charge, Y being a nucleophilic function chosen from a tertiary amine, an ether or a thioether.

Said polymer material comprises cavities referred to as molecularly imprinted cavities, the geometric and chemical configuration of which makes it possible to let the organophosphorus compounds to be detected penetrate into said polymer material.

### Receptor Group

Within the meaning of the present invention, the organophosphorus compounds are compounds of formula (W═)PL₁L₂L₂, in which W is O or S and at least one of the groups L₁, L₂, L₃ represents a leaving group.

The expression “leaving group” is understood to mean a labile nucleofugal group, i.e. a group capable of being substituted by a nucleophilic group during a nucleophilic attack. As examples of leaving groups, mention may especially be made of halogen atoms, in particular bromine, chlorine and fluorine atoms, and the groups AlkO—, AlkS— where Alk represents an alkyl group comprising from 1 to 6 carbon atoms, and the groups ArO—, ArS— where Ar represents an aryl group substituted by one or more NO₂ and/or Cl groups or that is unsubstituted.

The exposure of the receptor group to an organophosphorus compound results in the formation of a nucleofugal intermediate group —Z, resulting from the nucleophilic substitution of a labile ligand L₃ on the phosphorus atom with the nucleophilic function X.

Z may thus be a phosphate, phosphoramidate or thiophosphate group, i.e. a group resulting from the formation of an —O—P(═O), —N—P(═O), or —S—P(═O) covalent bond respectively.

An intramolecular nucleophilic attack by the function Y (ether (—O—), thioether (—S—) or tertiary amine (—N—)), on the carbon atom of the receptor group bearing the function Z (—O—P(═O), —N—P(═O), —S—P(═O)) follows in order to lead to the formation of a cyclized compound having a positive electronic charge located on the heteroatoms N, O or S of the function Y.

The reaction of the receptor group as defined above with an organophosphorus compound is illustrated in the scheme below.

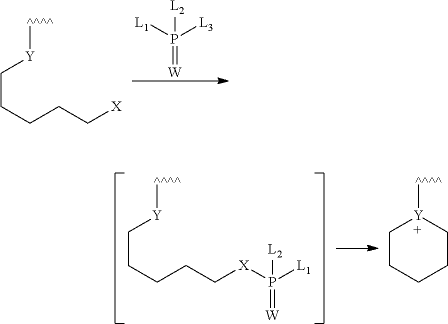

The generation of an electronic charge makes it possible to abruptly modify the electrostatic environment of the semiconductor material.

The expression “function Y capable of reacting with said group —Z in order to form a ring bearing a positive charge” is understood in particular to mean that the functions X and Y of the receptor group are in spatial proximity to one another, so as to enable intramolecular cyclization.

Preferably, the reaction of the nucleophilic function Y with the group —Z results in the formation of a 5- to 7-membered ring.

Preferably, the receptor group is a group of formula -A-L-X in which A is a group comprising a function Y, and L is a group —(CH₂)ₙ—, where n is an integer from 0 to 6. More preferably, n is an integer from 0 to 2.

Preferably, X is —CH₂OH.

Preferably, A is a group comprising a tertiary amine, in particular a nitrogen-containing heterocycle, more particularly a heterocycloalkyl group or a heteroaryl group.

Preferably, the receptor group is a group of formula (I):

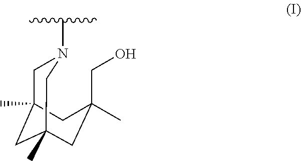 [SMILES: *N1C[C@@]2(C)CC(C)(CO)C[C@@](C)(C1)C2]

According to another preferred embodiment, the receptor group is a group of formula (II):

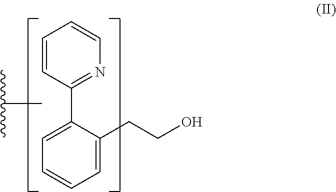

### Molecularly Imprinted Polymer Material

Generally, polymers referred to as molecularly imprinted polymers (MIPs) are robust biomimetic systems that make it possible to selectively capture a given molecule type.

Just like biological receptors, MIPs benefit from a great affinity and a good selectivity for given molecules. A priori, MIPs can be devised in the image of any molecule or family of functional molecules: thus, it is possible to envisage the synthesis of custom MIPs and more particularly of MIPs for target molecules for which no biological equivalent exists.

Due to their highly crosslinked chemical structure, MIPs have a very good thermal and chemical stability. Moreover, they have the advantage of being synthesized from low-cost reactants. The MIPs can be of various natures: organic, hybrid organic-inorganic or inorganic.

As summarized in the scheme illustrated in FIG. 1 and described in more detail below, the molecularly imprinted polymer (MIP) may be obtained according to a process comprising the steps of:
polymerization, using an initiator, and in the presence of a crosslinking agent, of one or more types of polyfunctional monomers (mf), in the presence of a molecule referred to as a molecular imprint which may be either directly the molecule to be detected, or a steric and chemical analog, and
extraction of the molecular imprint.

More particularly, during a first step referred to as the prearrangement step, the molecule referred to as the molecular imprint develops interactions with one or more functional monomers in a pore-forming solvent. These may be ionic or hydrophobic interactions or else hydrogen bonds.

During a second step referred to as the polymerization step, the addition of a crosslinking agent and of a polymerization initiator results in the formation of a synthetic matrix containing recognition sites specifically constructed around the imprint molecule.

During the third step referred to as the extraction step, the molecular imprint is eliminated using a suitable solvent: a polymer matrix is finally obtained that has cavities referred to as imprinted cavities, the geometric and chemical configuration of which is perfectly suitable for the fixation or reception of the molecules of interest, these cavities communicating with the external surface of the polymer material by means of channels.

Preferably, the polymer material comprising molecularly imprinted cavities is obtained by polymerization of an acrylic or methacrylic acid monomer.

According to one embodiment, the imprint molecule that makes it possible to obtain a selectivity of the polymer for the organophosphorus compounds is pinacolyl methylphosphonate.

The initiator may especially be 2,2′-azobis(2,4-dimethyl)valeronitrile.

The crosslinking agent may especially be ethylene glycol dimethacrylate.

According to one preferred variant, the polymer of acrylic or methacrylic acid is polymerized in the presence of at least one receptor molecule of formula R-A-L-X, in which R is a group which enables the receptor group to be incorporated in the structure of the imprinted polymer, A, L and X being as defined above.

Preferably, R is a group comprising a function CH₂═CH—, in particular a group CH₂═CH—(C₆H₄)—CH₂— or a group CH₂═CH—O—(CO)—(C₆H₄)—O—.

According to one preferred variant, the compound R-A-L-Y is a compound of formula (Ia) or (Ib):

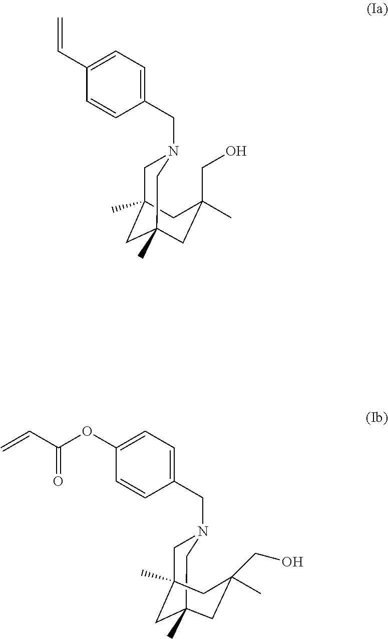 [SMILES: C=Cc1ccc(CN2C[C@@]3(C)CC(C)(CO)C[C@@](C)(C2)C3)cc1 ; C=CC(=O)Oc1ccc(CN2C[C@@]3(C)CC(C)(CO)C[C@@](C)(C2)C3)cc1]

Advantageously, all or some of the cavities of the molecularly imprinted polymer comprise at least one receptor group as defined in the present application, capable of reacting with the organophosphorus compound(s) to be quantified and/or detected.

Preferably, the molecularly imprinted polymer material is a film, the thickness of which is between 2 nm and 100 nm.

The invention also relates to a polymer material capable of being obtained according to a process comprising the steps of:
polymerization, using an initiator, and in the presence of a crosslinking agent, of one or more types of polyfunctional monomers (mf), in the presence of a molecule referred to as a molecular imprint which may be either directly the molecule to be detected, or a steric and chemical analog, and of a receptor molecule as defined in the present application, and
extraction of the molecular imprint.

### Electrical Device

The electrical device may be of resistor type, or else of field-effect transistor type.

When the electrical device is of the resistor type, the variation in intensity of the current between the source and drain electrodes, said variation in current intensity being caused by the production of positive charges during the cyclization of the receptor molecule when it comes into contact with the organophosphorus compounds, is, for example, detected and, optionally, measured at a given known voltage applied between the source and drain electrodes. This variation in current intensity gives the variation in conductance.

When the electrical device is of the transistor type, the semiconductor part is separated from the gate by a dielectric material.

Here again, the variation in the intensity of the source-drain current is, for example, detected and, optionally, measured at a given known voltage applied between the source and drain electrodes passing through the transistor. Since the intensity of the current is a function of the voltage of the gate, this then gives the transconductance of the semiconductor.

In the two cases, the variation in conductance or the variation in transconductance reveals the presence of organophosphorus compounds and is proportional to the concentration of organophosphorus compounds.

The semiconductor material acts as a transducer of the chemical signal to a signal of electrical nature. Indeed, when the reaction takes place between an organophosphorus molecule and the receptor bound to the polymer, the product of the reaction is a salt having a cation and an anion. The formation of this salt greatly disrupts the electrical properties of the semiconductor material on which the sensitive material is deposited. It is therefore the monitoring of one or more of the electrical properties of the semiconductor material that will reveal the detection of organophosphorus compounds.

The semiconductor material which plays the role of conduction channel is a semiconductor material, advantageously based on carbon, on silicon, on germanium, on zinc, on gallium, on indium, on cadmium or on an organic semiconductor material.

Preferably, the semiconductor material is constituted of silicon nanowire (s) and/or carbon nanotube(s).

More preferably, the semiconductor material is constituted of silicon nanowire(s) etched onto an SOI (silicon on insulator) surface.

In the case of organic semiconductor materials, the latter may be oligomers, polymers or small molecules. For example, they may be heterocyclic aromatic compounds such as thiophenes and derivatives thereof, preferably P3HT (poly-3-hexylthiophene), or polypyrroles and derivatives thereof, arylamines and derivatives thereof, preferably PTA (polytriarylamine), triarylamine-fluorene copolymers, isochromenones and derivatives thereof, heterocyclic macrocycles such as porphyrins, phthalocyanines and derivatives thereof. The organic semiconductor materials may also be aromatic polycyclic acenes and derivatives thereof, preferably anthracene or pentacene, arylenes and derivatives thereof, for example perylene, poly(para-phenylene), poly(para-phenylene vinylene) or polyfluorene. The organic semiconductor materials may also be polysilanes and derivatives thereof.

The electrodes may be metal electrodes, for example made of gold, silver, palladium, platinum, titanium, doped silicon, copper or nickel.

The simple structure of the device enables low-cost and large-scale production. In addition, due to this simple structure, the device may be of very small size, requiring little energy in order to function, favoring its portability.

Another subject of the invention relates to a multisensor comprising a device for detecting and/or quantifying organophosphorus compounds according to the present invention.

The term “multisensor” is understood to mean a device comprising several individual sensors assembled with one another, it being possible for these individual sensors to be provided with different sensitive materials and/or transducers.

### Polymer Material

Another subject of the present invention relates to a polymer material for detecting and/or quantifying at least one organophosphorus compound, said polymer material bearing at least one receptor group comprising:
a function X capable of reacting with the organophosphorus compound to be detected in order to form a group —Z, X being chosen from —CH₂OH, —CH₂NH₂, —CH₂SH; and
a function Y capable of reacting with said group —Z in order to form a ring bearing a positive charge, Y being a nucleophilic function chosen from a tertiary amine, an ether or a thioether;
   and
   said polymer material comprising cavities referred to as molecularly imprinted cavities, the geometric and chemical configuration of which is defined so as to let the organophosphorus compounds to be detected penetrate into said polymer material.

Preferably, the receptor group is a group of formula (I) or (II) as defined above.

Preferably, the polymer material is an acrylic polymer grafted by receptor groups as defined above.

Another subject of the present invention relates to a process for detecting and/or quantifying at least one organophosphorus compound, characterized in that it comprises the following steps:
placing the sample to be tested in liquid form or in gaseous form in the presence of a detecting and/or quantifying device as defined above,
demonstrating the positive charges generated by the reaction of the receptor group with the organophosphorus compound, by measuring the variation in resistance, in conductance or in transconductance of said device.

### DEFINITIONS

As they are used above, and throughout the description of the invention, the following terms, unless otherwise mentioned, should be understood as having the following meanings:

“Alkyl” denotes an aliphatic, linear, branched or cyclic hydrocarbon-based chain comprising from 1 to 12 carbon atoms, in particular from 1 to 6 carbon atoms. Branched means that one or more lower alkyl groups, such as methyl, ethyl or propyl, are bonded to a linear alkyl chain. “Lower alkyl” refers to an alkyl group comprising from 1 to 4 carbon atoms.

“Alkylene” refers to a substituted or unsubstituted, branched, linear or cyclic hydrocarbon-based chain comprising from 1 to 12 carbon atoms, resulting from the removal of two hydrogen atoms so as to form a divalent group. By way of example, mention may especially be made of methylene (—CH₂—) or 1,2-ethanediyl (—CH₂CH₂—).

“Aryl” refers to an aromatic group defined as a cyclic group that satisfies Hückel's rule, that is to say having a number of delocalized n electrons equal to (4n+2). By way of examples, mention may be made of cyclopentadienyl, phenyl, benzyl, biphenyl, phenylacetylene, pyrenyl and anthracenyl groups.

“Arylene” refers to an aryl group as defined above in which 2 hydrogen atoms have been removed in order to form a divalent group. By way of example, mention may be made of the phenylene group:

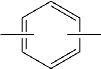

The “tertiary amine” function is understood within the meaning of the present application to mean a function in which the nitrogen atom is not bonded to any hydrogen atom. The nitrogen atom may thus be bonded to 3 carbon-based groups or else may be included in an aromatic system such as a pyridine.

An “ether” function is understood to mean a -Alk-O-Alk- or -Alk-O—Ar group, in which the term Alk denotes an alkyl or alkylene group and Ar denotes an aryl or arylene group, the alkyl, alkylkene, aryl and arylene groups being as defined above.

A “thioether” function is understood to mean an -Alk-S-Alk- or -Alk-S—Ar group in which the term Alk denotes an alkyl or alkylene group, and Ar denotes an aryl or arylene group, the alkyl, alkylene, aryl and arylene groups being as defined above.

The term “heterocycle” refers to a substituted or unsubstituted carboxylic group in which the cyclic part comprises at least one heteroatom such as O, N or S. The nitrogen and sulfur atoms may optionally be oxidized, and the nitrogen atom may optionally be substituted in an aromatic or non-aromatic ring. Within the meaning of the present description, the heterocycles comprise heteroaryl and heterocycloalkyl groups.

The term “heterocycloalkyl” refers to a cycloalkyl group in which one or more carbon atoms are replaced by at least one heteroatom such as —O—, —N— or —S—. As examples of heterocycloalkyl groups, mention may especially be made of pyrrolidinyl, pyrrolinyl, imidazolidinyl, imidazolinyl, pyrazolidinyl, pyrazolinyl, pyrazalinyl, piperidyl, piperazinyl, morpholinyl, thiomorpholinyl, tetrahydrofuranyl, dithiolyl, oxathiolyl, dioxazolyl, oxatriazolyl, pyranyl, oxazinyl, oxathiazinyl, and oxadiazinyl groups.

The term “heteroaryl” refers to an aromatic group containing from 5 to 10 carbon atoms in which one or more cyclic carbon atoms are substituted with one or more heteroatoms such as —O—, —N—, —S—. As examples of heteroaryl groups, mention may especially be made of pyrrolyl, furanyl, thienyl, pyrazolyl, imidazolyl, triazolyl, isothiazolyl, isoxazolyl, oxazolyl, oxathiolyl, oxadiazolyl, triazolyl, oxatriazolyl, furazanyl, tetrazolyl, pyridyl, pyrazinyl, pyrimidinyl, pyridazinyl, triazinyl, indolyl, isoindolyl, indazolyl, benzofuranyl, isobenzofuranyl, purinyl, quinazolinyl, quinolyl, isoquinolyl, benzoimidazolyl, benzothiazolyl, benzothiophenyl, thianaphthenyl, benzoxazolyl, benzisoxazolyl, cinnolinyl, phthalazinyl, naphthyridinyl, and quinoxalinyl groups. Included in this definition are the fused cyclic systems in which the aromatic ring is fused to a heterocycloalkyl group. By way of example of such fused cyclic systems, mention may especially be made of phthalamide, phthalic anhydride, indoline, isoindoline, tetrahydroisoquinoline, chromane, isochromane, chromene and isochromene groups.

The following examples illustrate the invention, without however limiting it. The starting products used are known products or products prepared according to known procedures.

### FIGURE

FIG. 1: FIG. 1 represents the synthesis of the polymer material according to the invention.

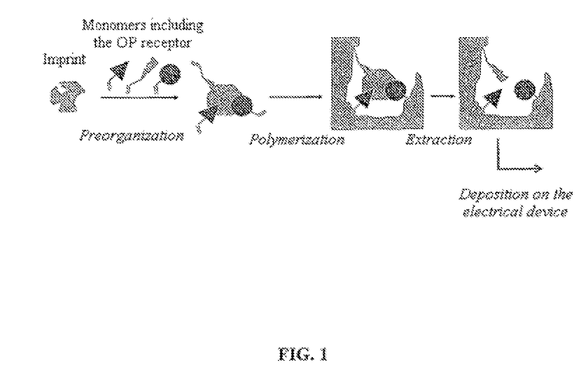

### EXAMPLES

### Example 1

### Synthesis of a Molecularly Imprinted Material a According to the Invention at the Surface of a Semiconductor Material (Silicon Nanowire(s))

A silicon nanowire(s) field-effect transistor is prepared from a p-doped (10¹⁵ B atom/cm³) SOT wafer. The silicon wire having dimensions 70 nm thick, 0.2 μm wide and 2 μm long is obtained by electron beam lithography and ion etching. The thickness of the dielectric material (SiO₂) is 140 nm. The source and drain Ti/Au (10/100 nm) electrodes are produced by electron beam lithography and lift-off. The degenerated silicon substrate is used as a gate electrode. The layer of molecularly imprinted polymer material is photocrosslinked at the surface of the semiconductor material as follows.

A solution containing two monomers of methacrylic acid (1 mmol) and the receptor selective for OPs (Ia), the structure of which is presented below (1 mmol), a crosslinking agent ethylene glycol dimethacrylate (5 mmol), a photoinitiator 2,2′-azobis(2,4-dimethyl)valeronitrile (60 mg), the molecular imprint pinacolyl methylphosphonate (0.25 mmol) is diluted with 2 mL of toluene. This reaction mixture is degassed via three vacuum-argon cycles, then 10 μL of this solution are deposited on the surface of the semiconductor material under nitrogen. The transistor is simultaneously subjected to the spin coater (30 sec, 3000 rpm) and to UV photoirradiation (30 sec). The sample is then rinsed with toluene and acetone and then placed in methanol overnight in order to extract the molecular imprint. The sample is then rinsed with anhydrous methanol and then dried under vacuum.

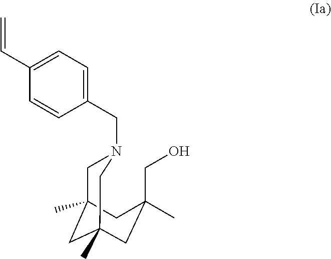 [SMILES: C=Cc1ccc(CN2C[C@@]3(C)CC(C)(CO)C[C@@](C)(C2)C3)cc1]

### Example 2

### Synthesis of a Molecularly Imprinted Material B According to the Invention at the Surface of a Semiconductor Material (Carbon Nanotubes)

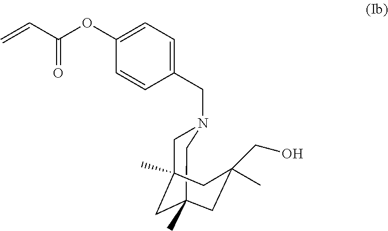 [SMILES: C=CC(=O)Oc1ccc(CN2C[C@@]3(C)CC(C)(CO)C[C@@](C)(C2)C3)cc1]

A carbon nanotube field-effect transistor is prepared by vaporization on a silicon wafer comprising a layer of native oxide (dielectric material having a thickness of 100 nm) and gold source and drain electrodes produced by electron beam lithography and lift-off. The electrodes are spaced apart by a 20 μm channel. A solution of single-walled carbon nanotubes (SWCNTs) dispersed in N-methylpyrrolidone (0.05 g/L dispersion of SWCNTs, subjected to ultrasounds for 90 min then centrifuged 2 times 1 hour at 14 000 rpm) is nebulized for 10 sec over the layer of the dielectric material between the electrodes. The degenerated silicon substrate is used as a gate electrode. The layer of molecularly imprinted polymer material is photocrosslinked at the surface of the semiconductor material as follows.

A solution containing two monomers of methacrylic acid (1 mmol) and the receptor selective for the OPs (Ib), the structure of which is presented above (1 mmol), a crosslinking agent trimethylolpropane trimethylmethacrylate (5 mmol), a photoinitiator 2,2-dimethoxy-2-phenylacetophenone (100 mg), the molecular imprint pinacolyl methylphosphonate (0.25 mmol) is diluted with 2 mL of a mixture of toluene/acetonitrile (1:1) solvents. This reaction mixture is degassed via three vacuum-argon cycles (vacuum=50 mTorr, argon under atmospheric pressure), then 10 μL of this solution are deposited at the surface of the semiconductor material under nitrogen. The transistor is simultaneously subjected to the spin coater (30 sec, 3000 rpm) and to UV photoirradiation (30 sec). The sample is then rinsed with toluene and acetone and then placed in methanol overnight in order to extract the molecular imprint. The sample is then rinsed with anhydrous methanol and then dried under vacuum.

### Example 3

### Detection of Diphenyl Chlorophosphate (DPCP) and of Interferents (Organic Solvents, Bases and Acids) by a Silicon Nanowire(s) Transistor Comprising at its Surface a Thin Film of Molecularly Imprinted Material a Prepared According to Example 1

Between each exposure, a new sensor prepared according to example 1 is used. The source (S), drain (D) and gate (G) electrodes are subjected to the following potentials: V_{DS}=−1 V and V_{GS}=+2 V. The current I_{DS} is then measured as a function of the time. The current I_{DS} is stable when the sensor is in air only (I_{DS}=10⁻¹⁰ A). After exposing the transistor to vapors of organophosphorus compounds (500 ppb of diphenyl chlorophosphate in dry synthetic air for 3 minutes), the current I_{DS} increases very rapidly by a factor of greater than 100 in less than one minute.

On the other hand, when a sensor prepared according to example 1 is subjected to the following compounds, variations of the current I_{DS} of less than 5% are observed:
cyclohexane at a concentration of 20 000 ppm in dry synthetic air, for 3 minutes;
dichloromethane at a concentration of 530 000 ppm in dry synthetic air, for 3 minutes
acetonitrile at a concentration of 130 000 ppm in dry synthetic air, for 3 minutes
triethylamine at a concentration of 75 000 ppm in dry synthetic air, for 3 minutes
propionic acid at a concentration of 20 000 ppm in dry synthetic air, for 3 minutes.

Studying the prior art demonstrates the difficulty in having a selective sensor for organophosphorus compounds. As the response of the sensor is extremely weak with respect to interferents and very high with respect to diphenyl chlorophosphate, this example reveals the extreme sensitivity and excellent selectivity of the sensor for organophosphorus compounds.

### Example 4

### Detection of Diphenyl Chlorophosphate (DPCP) by a Carbon Nanotube Transistor Comprising at its Surface a Thin Film of Molecularly Imprinted Material B Prepared According to Example 2

Between each exposure, a new sensor prepared according to example 2 is used. The source (S), drain (D) and gate (G) electrodes are subjected to the following potentials: Vₐₛ=−0.5 V and V_{Gs}=+10 V. The current I_{DS} is then measured as a function of the time. The current I_{DS} is stable when the sensor is in air only (I_{DS}=10⁻¹⁰ A). After exposing the transistor to vapors of organophosphorus compounds (500 ppb of diphenyl chlorophosphate in dry synthetic air for 3 minutes), the current I_{DS} increases very rapidly by a factor of greater than 10 in less than one minute. It is observed experimentally that the variation of the conductance or the variation of the transconductance reveals the presence of organophosphorus compounds and is proportional to the number of receptors that have reacted with the OPs. The response (variation of the positive charges) is even faster and larger when the concentration of Ops is high. By producing a calibration curve, a quantification of the OPs may be achieved.

1. A device comprising:
an electrical device comprising a source electrode and a drain electrode separated by a semiconductor material, and
a device for detecting positive charges between the two electrodes,
wherein said device comprises a surface of the semiconductor material to be in contact with a polymer material bearing at least one receptor group comprising:
a function X capable of reacting with the organophosphorus compound to be detected in order to form an intermediate group —Z, wherein X is —CH₂OH, —CH₂NH₂, or —CH₂SH; and
a function Y capable of reacting with said group —Z in order to form a ring bearing a positive charge, wherein Y is a nucleophilic function of a tertiary amine, an ether or a thioether; wherein said polymer material comprises cavities referred to as molecularly imprinted cavities, the geometric and chemical configuration of which makes it possible to allow the detectable organophosphorus compounds to penetrate into said polymer material.

2. The device according to claim 1, wherein the receptor group is a group of formula wherein L is a group —(CH₂)ₙ—, where n is an integer from 0 to 6, and wherein A is a group comprising a function Y.

3. The device according to claim 1, wherein X is —CH₂OH.

4. The device according to claim 2, wherein A is a group comprising a tertiary amine.

5. The device according to claim 4, wherein A is a nitrogen-containing heterocycle.

6. The device according to claim 1, wherein the receptor group is a group of formula (I):

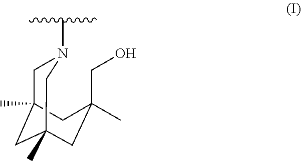 [SMILES: *N1C[C@@]2(C)CC(C)(CO)C[C@@](C)(C1)C2]

7. The device according to claim 1, wherein the receptor group is a group of formula (II):

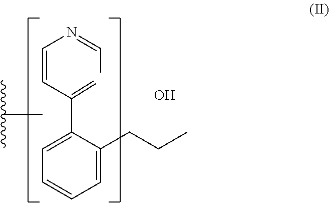

8. The device according to claim 1, wherein the polymer material comprising molecularly imprinted cavities is a obtained by polymerization product of acrylic or methacrylic acid monomers.

9. The device according to claim 8, wherein the polymerization of acrylic or methacrylic acid is carried out in the presence of at least one compound of formula R-A-L-X,
wherein R is CH₂═CH—,
wherein A is a group comprising a function Y, and L is a group —(CH)ₙ—, where n is an integer from 0 to 6 and X being as defined in claim 2.

10. The device according to claim 1, wherein the semiconductor material is silicon nanowire(s) carbon nanotube(s), or combinations thereof.

11. The device according to claim 1, wherein said electrical device is of field-effect transistor type.

12. A multisensor comprising a device for detecting and/or quantifying at least one organophosphorus compound according to claim 1.

13. A polymer material for detecting and/or quantifying at least one organophosphorus compound, said polymer material bearing at least one receptor group comprising:
a function X capable of reacting with the organophosphorus compound to be detected in order to form an intermediate group —Z, wherein X is —CH₂OH, —CH₂NH₂, or —CH₂SH; and
a function Y capable of reacting with said group —Z in order to form a ring bearing a positive charge, wherein Y is being a nucleophilic function of a tertiary amine, an ether or a thioether; and
wherein said polymer material comprises cavities referred to as molecularly imprinted cavities, the geometric and chemical configuration of which is are defined so as to allow the detectable organophosphorus compounds to penetrate into said polymer material.

14. The polymer material according to claim 13, wherein the receptor group is a group of formula (I)

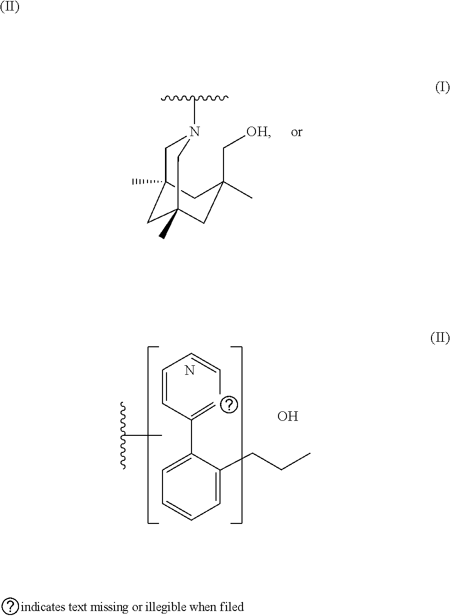

15. A process for detecting and/or quantifying at least one organophosphorus compound, comprising:
placing a sample to be tested in liquid form or in gaseous form in the presence of a detecting and/or quantifying device according to claim 1,
demonstrating the positive charges generated by the reaction of the receptor group with the organophosphorus compound, by measuring the variation in resistance, in conductance or in transconductance of said device.

16. A process for detecting and/or quantifying at least one organophosphorus compound, comprising:
placing a sample to be tested in liquid form or in gaseous form in the presence of a multisensor according to claim 12,
demonstrating the positive charges generated by the reaction of the receptor group with the organophosphorus compound, by measuring the variation in resistance, in conductance or in transconductance of said device.
